(19) United States
(12) Patent Application Publication
(10) Pub. No.: US 2009/0319293 A1
(43) Pub. Date: Dec. 24, 2009

(54) VIRTUAL INVENTORY SYSTEM AND METHODS FOR COVERED ENTITIES

(75) Inventors: Grover C. Lee, Lincoln, CA (US); David A. Valerio, Auburn, CA (US)

Correspondence Address: JOHN P. O'BANION;O'BANION & RITCHEY LLP, 400 CAPITOL MALL SUITE 1550, SACRAMENTO, CA, 95814, US

(73) Assignee: AMERICAN HEALTH CARE, Rocklin, CA (US)

(21) Appl. No.: 12/144,546

(22) Filed: Jun. 23, 2008

(51) Int. Cl. G06Q 50/00; G06Q 10/00

(52) U.S. Cl. 705/2; 705/28

(57) ABSTRACT

A virtual transaction process and system providing a mechanism for a covered entity to maintain a virtual inventory with a contracted pharmacy for dispensing medication for the covered entity's employees. An administrative entity is provided that adjudicates claims with the contracted pharmacy, and compiles a utilization report for the prescription medication manufacturer and covered entity to effect a chargeback credit from the manufacturer and payment between the covered entity and contracted pharmacy.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to dispensing of prescription medicine, and more particularly to a virtual inventory tracking system for prescription medicine.

2. Description of Related Art

A covered entity (CE), such as a hospital, typically maintains an on-premise pharmacy that has sufficient capacity to service its in-patients. The CE purchases pharmaceuticals from a wholesaler who in turn obtains, or has obtained, the pharmaceuticals from a manufacturer.

Some CEs, such as non-profit facilities, are provided with deep discounts on pharmaceuticals as compared to retail prices. These discounts are generally provided directly by the manufacturer. However, because the CE obtains the pharmaceuticals from a wholesaler, the CE does not realize the full amount of the manufacturer's discount because a portion of the discount is lost due to the wholesaler's “cut”.

In many cases, CEs also provide a benefit where the employees can have prescriptions filled by the hospital pharmacy. However, inventory management often becomes burdensome for the hospital pharmacies that service both in-patients and employees.

Another factor is a large number of these entities are small regional hospitals; thus developing a pharmacy facility capable of accommodating the filling of employee prescriptions is an impractical option for often financially strapped CEs.

Additionally, the national shortage of pharmacists makes it near impossible for these small regional hospitals to acquire and retain staff due to aggressive competition for available pharmacists. Accordingly, an object of the present invention is a mechanism to remove the wholesaler cost from the pharmaceutical supply chain so that the CE can realize a cost savings. Another aspect is an employee prescription benefit dispensing system and method that provides greater savings to the non-profit entity. A further aspect is an inventory management system that reduces the inventory management burden on hospital pharmacies resulting from the usual method of filling employee prescriptions. At least some of these objects will be met in the following description.

### BRIEF SUMMARY OF THE INVENTION

An aspect of the present invention is to eliminate a CE's need for physical inventory management for employee prescriptions. In one embodiment of the present invention, a “virtual inventory”, or virtual pharmacy, is created, which is separate from the hospital's in-patient pharmacy, to fill the prescriptions of the CE employees. The virtual inventory may be an in-patient pharmacy inside of the hospital (created by managing a virtually separate inventory), an outpatient pharmacy owned by the CE, or an independently operated retail pharmacy. The hospital may order pharmaceuticals from its wholesaler at an agreed-upon price, wherein the wholesaler ships the goods to the pharmacy at no cost, and the hospital pays the wholesaler for the goods at the contract price. Thus, there is no change in the physical supply chain used by the virtual inventory, yet the virtual inventory fills the CE employees' prescriptions instead of the in-patient pharmacy. This process is conducted by an administrative entity hired by the CE.

An aspect of the invention is method of dispensing a prescription for a covered entity. The method includes the steps of providing a contracting pharmacy having inventory to fill prescriptions for a covered entity, receiving a transmission from the contracting pharmacy, the transmission comprising a prescription pharmaceutical claim, adjudicating the pharmaceutical claim and transmitting the adjudicated claim back to the contracting pharmacy, compiling a utilization report for the pharmaceutical claim, sending the utilization report to a pharmaceutical manufacturer for payment of a chargeback credit, payment of the chargeback credit, and sending the utilization report to the covered entity for payment of contract pharmaceutical price plus administrative fees.

In a preferred embodiment, the method also includes receiving the chargeback credit from manufacturer and payment from covered entity, and issuing payment to the contracted pharmacy. Often, the payment issued to the contracted pharmacy will comprise the WAC prescription cost plus a pharmacy dispensing fee, less any co-pay collected by the contracted pharmacy. Alternatively payment of the chargeback credit and contract pharmaceutical price to the contracted pharmacy may be facilitated (e.g. by an administrative entity, wherein an administrative fee is received from the covered entity.)

Adjudicating the claim may comprise verifying client eligibility and claim data validity, and determining co-pay amount, among other elements.

Generally, the pharmaceutical claim is transmitted with an identifying number called a national drug code number, or NDC. The pharmaceutical claim is generally adjudicated by comparing the transmitted claim against one or more databases of stored NDC codes. Furthermore, the utilization report may be generated and separated according to the covered entity, the contracted pharmacy, patient, and NDC.

In another embodiment of the current aspect, providing a contracting pharmacy comprises providing an administrative entity in communication with the covered entity, at least one contracting pharmacy, and at least one pharmaceutical manufacturer, and facilitating an agreement via the administrative entity between the covered entity and at least one contracting pharmacy for dispensing prescriptions for the covered entity. Preferably, the administrative entity adjudicates the pharmaceutical claim and compiles and sends the utilization report for the pharmaceutical claim to the manufacturer and covered entity.

Another aspect is a method of dispensing a medication for a covered entity. The method includes the steps of providing an administrative entity in communication with the covered entity, at least one contracting pharmacy, and at least one pharmaceutical manufacturer, arranging for at least one contracting pharmacy to dispense medication for the covered entity, receiving a transmission from the contracting pharmacy to the administrative entity, the transmission comprising a prescription claim for a medication, adjudicating the prescription claim, transmitting the adjudicated claim back to the contracting pharmacy, and compiling a utilization report for the prescription claim for payment of a chargeback credit from a manufacturer of the medication and reimbursement from the covered entity to the contracting pharmacy.

In one embodiment of the sending the utilization report to the medication manufacturer for payment of a chargeback credit, and sending the utilization report to the covered entity for payment of contract medication price plus administrative fees.

The method may further include facilitating payment of the chargeback credit and contract medication price to the contracted pharmacy via the administrative entity, and receiving an administrative fee from the covered entity.

In one embodiment, the claim is transmitted to and from the administrative entity as an NDC. The claim may be adjudicated by comparing the transmitted claim against one or more databases of stored price lists by NDC codes, the databases being maintained by the administrative entity.

In an alternative embodiment, the utilization report is sent to the covered entity for payment of contract medication price plus administrative fees, wherein the covered entity facilitates payment of chargeback credit via a wholesaler.

Another aspect is a virtual inventory system for dispensing a prescription medication for a covered entity, comprising a communication link coupled to the covered entity, one or more medication manufacturers, and one or more contracting pharmacies, a processor coupled to the communication link, the processor configured to adjudicate a prescription medication claim transmitted from a contracting pharmacy to facilitate dispensing of the prescription medication at the contracting pharmacy and compile a utilization report for the prescription medication claim, and one or more databases coupled to the processor, the databases comprising data used in adjudicating the claim and compiling a utilization report, wherein the utilization report facilitates a payment of a chargeback credit from the manufacturer of the prescription medication and payment of a contract prescription price plus administrative fees from the covered entity to the contracting pharmacy.

In an exemplary embodiment of the current aspect, the one or more databases comprise lists of NDC numbers, wherein the processor adjudicates the pharmaceutical claim by comparing the transmitted claim against one or more databases of stored NDC codes. The processor may also generate the utilization report according to the covered entity, the contracted pharmacy, patient, and NDC code.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 illustrates a flowchart for a manufacturer-direct virtual transaction process in accordance with the present invention.

FIG. 2 shows an alternative embodiment of the manufacturer-direct virtual transaction process of FIG. 1.

FIG. 3 illustrates a method of dispensing a medication for a covered entity using a virtual inventory.

FIG. 4 shows a flowchart for a virtual transaction process in wherein the wholesaler processes the chargeback in accordance with the present invention

FIG. 5 illustrates a schematic diagram of a system for providing a virtual inventory of medication for a covered entity in accordance with the present invention.

### DETAILED DESCRIPTION OF THE INVENTION

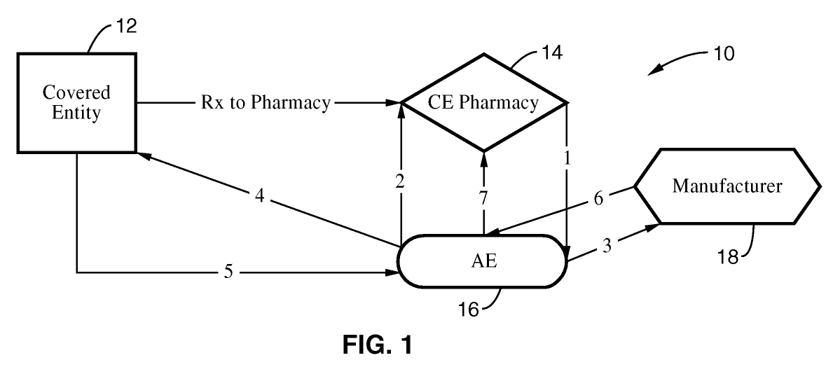

Referring more specifically to the drawings, for illustrative purposes the present invention is embodied in the system(s) and method(s) generally shown in FIG. 1 through FIG. 5. It will be appreciated that the apparatus may vary as to configuration and as to details of the parts, and that the method may vary as to the specific steps and sequence, without departing from the basic concepts as disclosed herein.

FIG. 1 illustrates an exemplary flowchart for a virtual transaction process 10 direct with the manufacturer in accordance with the present invention. First, the covered entity (CE) 12 will forward the prescription to the contracted CE pharmacy 14 that is specified to carry the inventory of medication for CE employees. The contracted pharmacy, or CE pharmacy 14, may be any facility with sufficient inventory to cover the inventory demands, such as a third party retail pharmacy, or a combination of retail pharmacies. Preferably, the CE pharmacy 14 is a local/community pharmacy that can provide accessibility to CE employees and will agree to a dispensing fee without marking up the pharmaceutical cost.

The CE 12 itself may include, but is not limited to, any facility to which a manufacturer 18 provides a discount over retail price, e.g. regional hospitals, HMOs (profit or nonprofit), stand-alone clinics, VA facilities, or the like.

In the first step of the virtual transaction process 10, the CE pharmacy transmits the prescription (for the covered entity employee or patient) to an administrative entity (AE) 16 (arrow 1), which is configured to adjudicate the prescription claims and interface between the CE 12, CE Pharmacy 14, and manufacturer 18.

The AE 16 is preferably equipped with a real-time, online claims processing computer system to provide centralized telecommunications for direct communication with a network of pharmacies 14 by telephone carrier lines, internet, or other remote connection means available in the art. The contracted pharmacy 14 generally enters the prescription data into their internal system through either a personal computer, terminal, or other point of sale device. The pharmacy 14 may have dial-up service that transmits over phone lines, such as currently available through a third party carrier (switch vendor), e.g. Envoy and National Data Center. The pharmacy 14 may also have a computer system and software that transmits the prescription over the internet to the AE 16. Adjudication of the claim may also be performed through a third-party switch vendor as detailed above.

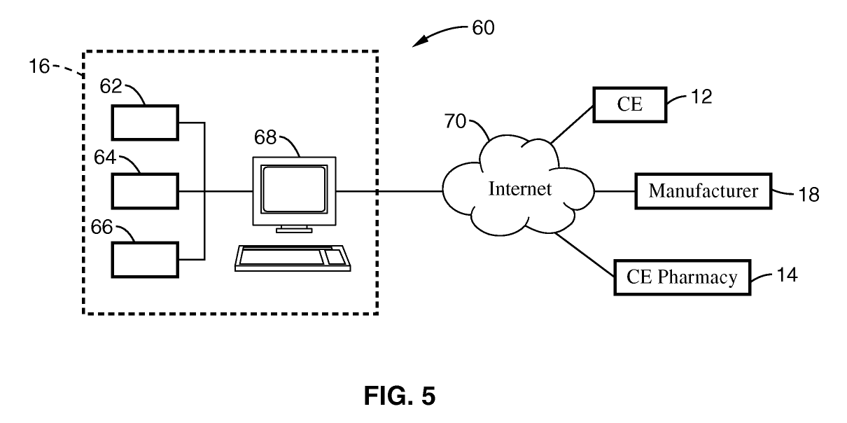

FIG. 5 illustrates a system 60 for providing a virtual inventory of medication for a covered entity. The AE 16 comprises a processor 68 having a communication link 70 (e.g. internet connection) with the CE 12, manufacturers 18, and available contract pharmacies 14. The processor 68 may be coupled to a series of databases 62, 64, and 66 having data (e.g. NDC codes), manufacturer lists, third-party pharmacies, etc. that are used in adjudicating claims and compiling utilization reports.

The prescription is transmitted to the AE 16 in a format that is compliant with National Council for Prescription Drug Programs (NCPDP) standards. The Health Insurance Portability & Accountability Act of 1996 (HIPAA) requires that the NCPDP format be used for the electronic submission of retail pharmacy claims. The incoming claims are first passed through an NCPDP translator and stored in a Flat-File-Format (FFF). ViPS programs then pass the FFF data through a series of editing programs and produce reports that list the claims included in the transmission and identify any errors in the submitted data.

Each listed drug product listed is assigned a unique 10-digit, 3-segment number. This number, known as the NDC (national drug code), identifies the labeler, product, and trade package size. The first segment, the labeler code, is assigned by the FDA. A labeler may be any firm that manufactures (e.g. manufacturer 18), or distributes the drug. The second segment, the product code, identifies a specific strength, dosage form, and formulation for a particular firm. The third segment, the package code, identifies package sizes and types. The various lists of NDC codes are maintained and updated by the FDA.

The AE 16 adjudicates the claim back to the CE pharmacy with eligibility, drug coverage, and co-pay amount due (arrow 2). Adjudication, for purposes of the following description, is defined as the role of the AE's authority in determining the suitability (acceptance or rejection) of the claim tendered by the pharmacist. The AE 16 may have one or more databases storing NDC codes, membership plan information, etc. for adjudicating the claim. A plan sponsor or insurer will generally have contracted with the pharmacist and defined the basis or the terms and conditions of reimbursement for dispensing a drug.

Prior to adjudication, each claim may be edited by computer (at the AE 16) for compliance to NCPDP standards for data form and completeness. This may then be followed by a confirmation of member eligibility using the AE's computer membership files. The adjudication process is then invoked according to the member's benefit plan parameters. Such parameters generally comprise listing of allowable drugs or supplies, quantity and dosage allowed, member's co-insurance or “co-pay”, member's contribution due to deductible or capitation considerations, if any member's allowance for refills, etc. These parameters may be stored in a plan file residing in the one or more databases maintained by the AE 16, or may be available online.

The adjudicated claim is adjudicated via a series of edits, and is transmitted in real-time or batch to the CE pharmacy 14 for verification of client eligibility (e.g. determine whether patient is part of the covered entity), verification of claim data validity (e.g. verify coverage of drug due to formulary restrictions, DESI status, obsolete dates and rebate closures, perform on-line duplicate services detection and drug caps, provider prospective drug utilization review (DUR), and detection of conflicts), price the claim and provide co-pay and reimbursement amounts.

The AE 16 compiles a computer-generated utilization report (UR) by covered entity (CE 12), contracted pharmacy (CE pharmacy 14), and NDC, along with supporting detail. The UR is sent to the manufacturer 18 for chargeback credit to the CE12 (arrow 3).

The AE 16 sends the report with detail and billing to the CE 12 for payment of claims at contract price (the manufacturer's discounted price to CE) plus and administrative fee to the AE 16 (arrow 4). The CE 12 then sends payment (contract price+AE fee) to the AE 16 (arrow 5).

The manufacturer 18 issues a credit to AE 16 (chargeback credit) for the difference between the WAC (wholesaler's acquisition cost) and the contract price (arrow 6). The WAC is the price generally charged by wholesalers to retail pharmacies.

Finally, the AE 16 pays the CE Pharmacy 14 the WAC price that it paid for the medication, in addition to a dispensing fee for the CE Pharmacy's services.

Wholesaler distribution is undisturbed in the above description because the wholesaler still supplies the CE pharmacy in the same fashion that it traditionally does. The above virtual process may be used to support any non-retail pharmacy transaction, with the exception of in-patient drug dispensing.

It is appreciated that a number of variations of the method illustrated in FIG. 1 may be implemented, particularly to the timing of transactions between the manufacturer 18, CE pharmacy 14, CE 12, and AE 16. For example, the AE 16 may make payment to the CE pharmacy 14 (arrow 7) prior to receiving the chargeback credit from the manufacturer 18 (arrow 6) and the payment from the CE 12 (arrow 5). The chargeback credit (arrow 6) from the manufacturer 18 may also be simultaneous with or prior to the payment from the CE 12 (arrow 5). In addition, the utilization report to the CE (arrow 4) may be simultaneous with or prior to the utilization report to the manufacturer 18 (arrow 3).

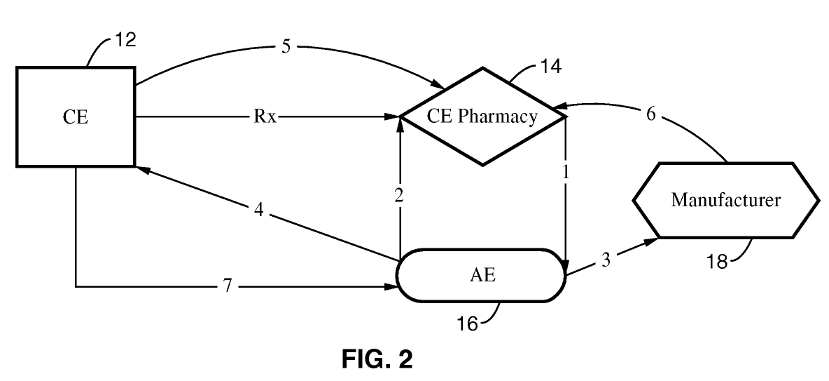

In an alternative embodiment shown in FIG. 2, the chargeback credit from the manufacturer 18 and the payment from the CE 12 may be routed directly to the CE pharmacy 14 (arrows 5 and 6), with the CE 12 paying just the administrative fee to the AE 16 (arrow 7). In this embodiment, the AE only facilitates payment from the manufacturer 18 and covered entity 12 to the pharmacy 14.

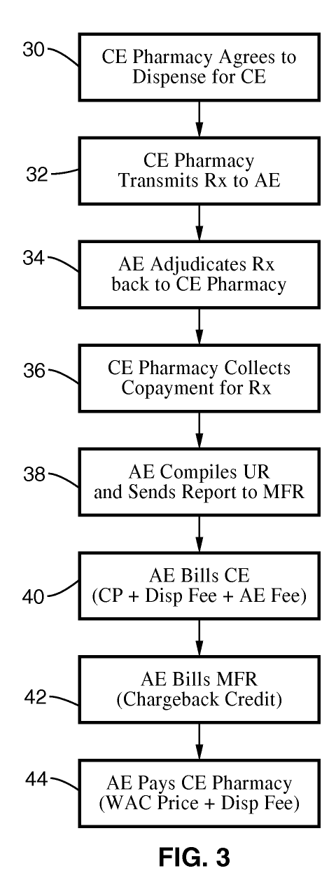

Another aspect of the invention is a transaction mechanism that results in a cost savings to the CE by using the contracted pharmacy to fill employees' prescriptions. In one embodiment, the cost savings is achieved by carrying out the method illustrated in FIG. 3.

First, in step 30, the CE 12 arranges with a third-party CE pharmacy to dispense pharmaceuticals to CE employees. The AE 16 may facilitate selection of the contracted CE pharmacy 14 from a list of retail pharmacies that have been compiled by the AE 16 to dispense pharmaceuticals.

The CE pharmacy 14 and CE 12 will generally agree on a dispensing fee (e.g., $5), which may be prearranged or negotiated between the parties. Generally, the CE pharmacy has a certain inventoried pharmaceutical for which it was billed (e.g., $100) by the wholesaler 18 (e.g. at WAC price). Also, the manufacturer will often grant a discounted price (e.g., $60) to the CE for the pharmaceutical.

Under this arrangement, the CE employee will take his/her prescription to the CE pharmacy 14 to fill the prescription. The CE pharmacy 14 transmits the prescription to the AE 16 in step 32. In step 34, the AE 16 then adjudicates the prescription claim back (e.g. in real time) to the CE pharmacy 14 with eligibility, drug coverage, and co-payment amount due from the CE employee. In step 36, the CE pharmacy 14 collects the co-payment (e.g., $15), if any, from the CE employee and dispenses the pharmaceutical to the CE employee. The CE pharmacy 14 will reorder the dispensed pharmaceutical according to its established inventory levels from its wholesaler.

In step 38, the AE 16 compiles a utilization report (UR) by CE, CE Pharmacy, patient, and NDC with supporting detail and sends the report to the pharmaceutical manufacturer 18 for a particular chargeback credit associated with the filled prescription.

The AE 16 then sends a report and bills CE 12 in step 40. The CE's bill will typically be the manufacturer's discount price (e.g., $60) plus the CE pharmacy's agreed-upon dispensing fee (e.g., $5), generating a total (e.g., $65). The AE 16 also bills CE 12 for its administrative fee (e.g., $3 per transaction).

In step 42, the AE 16 bills the manufacturer 18 for the chargeback credit, or the difference between the CE pharmacy's acquisition cost (e.g., $85—which is the CE pharmacy's $100 WAC less the $15 co-payment from patient) and the discounted price to the CE (e.g., $60)), resulting in a billing to the manufacturer (e.g., $25 chargeback credit). Step 42 may be performed at the same time as step 38 when the UR is sent to the manufacturer 18.

In the final step, the AE 16 pays the sum of the amounts collected from step 40 and step 42 (minus the AE administrative fee) to the CE pharmacy 14 (e.g., $90). In the above example, the CE pharmacy 14 has received the equivalent of $105 ($65 from step 40, $25 from step 42, and the $15 co-payment from step 36). Thus, the CE pharmacy 14 has received a $5 dispensing fee and the cost they paid for the pharmaceutical.

As detailed above, the timing of the above steps and routing of payment, may be varied accordingly. It is also appreciated that the above can be used with discharged patients and out-patients as well as CE employees, provided it is acceptable under the terms of the manufacturer contract.

The above method illustrated in FIG. 3 has a number of benefits. First, the manufacturer 18 increases its market share and volume by giving a discount to the CE 12. The CE pharmacy 14 increases its “foot traffic” and, hence, customer base. The CE employee is charged a lower co-payment by using the CE pharmacy 14 than if he or she went to another pharmacy. More importantly, the CE attains the benefit of a virtual pharmacy without having to allocate the space, inventory, and personnel required to support employee or outpatient prescription needs. Reconciliation of prescriptions is on a per unit basis eliminating the need for accumulation of utilization into a stock package amount.

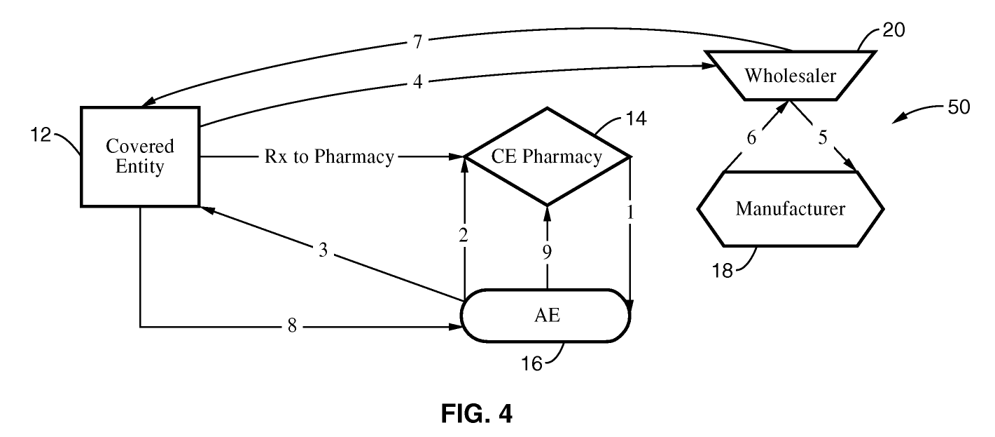

FIG. 4 illustrates an alternative embodiment of a virtual transaction process 50 wherein the CE payments are processed through wholesaler/PBM 20 in accordance with the present invention. In process 50, the CE pharmacy 14 transmits the prescription to the AE16 (arrow 1) and the AE 16 adjudicates the claim back to the CE pharmacy 14 (arrow 2) in the same manner as the process detailed in FIG. 1. However, the utilization report (UR) is not sent to the manufacturer 18, but rather just to the CE 12 (arrow 3). The CE 12 then uses the UR to request chargeback from the wholesaler 20 (arrow 4). The wholesaler 20 then requests a chargeback credit from the manufacturer 18 (arrow 5), and the manufacturer 18 issues the chargeback payment to the wholesaler 20 (arrow 6). The wholesaler 20 then issues a chargeback credit (WAC—contract price) to the CE 12.

Although the description above contains many details, these should not be construed as limiting the scope of the invention but as merely providing illustrations of some of the presently preferred embodiments of this invention.

Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A method of dispensing a prescription for a covered entity, comprising:
providing a contracting pharmacy having inventory to fill prescriptions for a covered entity;
receiving a transmission from the contracting pharmacy, the transmission comprising a prescription pharmaceutical claim;
adjudicating the pharmaceutical claim;
transmitting the adjudicated claim back to the contracting pharmacy;
compiling a utilization report for the pharmaceutical claim;
sending the utilization report to a pharmaceutical manufacturer for payment of a chargeback credit; and
sending the utilization report to the covered entity for payment of contract pharmaceutical price plus administrative fees.

2. A method as recited in claim 1, further comprising:
receiving the chargeback credit from manufacturer and payment from covered entity; and
issuing payment to the contracted pharmacy.

3. A method as recited in claim 2, wherein the payment issued to the contracted pharmacy comprises the WAC prescription cost plus a pharmacy dispensing fee, less any co-pay collected by the contracted pharmacy.

4. A method as recited in claim 1, further comprising:
facilitating payment of the chargeback credit and contract pharmaceutical price to the contracted pharmacy; and
receiving an administrative fee from the covered entity.

5. A method as recited in claim 1, wherein adjudicating the claim comprises:
verifying client eligibility;
verifying of claim data validity; and
determining co-pay amount.

6. A method as recited in claim 5, wherein the pharmaceutical claim is transmitted with an NDC.

7. A method as recited in claim 6, wherein the pharmaceutical claim is adjudicated by comparing the transmitted claim against one or more databases of stored NDC codes.

8. A method as recited in claim 6, wherein the utilization report is generated according to the covered entity, the contracted pharmacy, patient, and NDC.

9. A method as recited in claim 1, wherein providing a contracting pharmacy comprises:
providing an administrative entity in communication with the covered entity, at least one contracting pharmacy, and at least one pharmaceutical manufacturer; and
facilitating an agreement via the administrative entity between the covered entity and at least one contracting pharmacy for dispensing prescriptions for the covered entity.

10. A method as recited in claim 9, wherein the administrative entity adjudicates the pharmaceutical claim and compiles and sends the utilization report for the pharmaceutical claim to the manufacturer and covered entity.

11. A method of dispensing a medication for a covered entity, comprising:
providing an administrative entity in communication with the covered entity, at least one contracting pharmacy, and at least one pharmaceutical manufacturer;
arranging for at least one contracting pharmacy to dispense medication for the covered entity;
receiving a transmission from the contracting pharmacy to the administrative entity, the transmission comprising a prescription claim for a medication;
adjudicating the prescription claim;
transmitting the adjudicated claim back to the contracting pharmacy; and
compiling a utilization report for the prescription claim for payment of a chargeback credit from a manufacturer of the medication and reimbursement from the covered entity to the contracting pharmacy.

12. A method as recited in claim 11, further comprising:
sending the utilization report to the medication manufacturer for payment of a chargeback credit; and
sending the utilization report to the covered entity for payment of contract medication price plus administrative fees.

13. A method as recited in claim 11, further comprising:
receiving the chargeback credit from manufacturer and payment from covered entity; and
issuing payment to contracted pharmacy.

14. A method as recited in claim 13, wherein the payment issued to the contracted pharmacy comprises the WAC prescription cost plus a pharmacy dispensing fee, less any co-pay collected by the contracted pharmacy.

15. A method as recited in claim 11, further comprising:
facilitating payment of the chargeback credit and contract medication price to the contracted pharmacy via the administrative entity; and
receiving an administrative fee from the covered entity.

16. A method as recited in claim 11, wherein adjudicating the claim comprises:
verifying client eligibility;
verifying of claim data validity; and
determining co-pay amount.

17. A method as recited in claim 16, wherein the claim is transmitted to and from the administrative entity with an NDC.

18. A method as recited in claim 18, wherein the claim is adjudicated by comparing the transmitted claim against one or more databases of stored NDC codes;
said databases being maintained by the administrative entity.

19. A method as recited in claim 11:
wherein the utilization report is sent to the covered entity for payment of contract medication price plus administrative fees;
wherein the covered entity facilitates payment of chargeback credit via a wholesaler.

20. A virtual inventory system for dispensing a prescription medication for a covered entity, comprising:
a communication link coupled to the covered entity, one or more medication manufacturers, and one or more contracting pharmacies;
a processor coupled to the communication link;
the processor configured to adjudicate a prescription medication claim transmitted from a contracting pharmacy to facilitate dispensing of the prescription medication at the contracting pharmacy;
the processor further configured to compile a utilization report for the prescription medication claim; and
one or more databases coupled to the processor, the databases comprising data used in adjudicating the claim and compiling a utilization report;
utilization report facilitating a payment of a chargeback credit from the manufacturer of the prescription medication and payment of a contract prescription price plus administrative fees from the covered entity to the contracting pharmacy.

21. A system as recited in claim 20:
wherein the one or more databases comprise lists of NDC codes;
wherein the processor adjudicates the pharmaceutical claim by comparing the transmitted claim against one or more databases of stored NDC codes.

22. A system as recited in claim 20, wherein the processor generates the utilization report according to the covered entity, the contracted pharmacy, patient, and NDC.
